(19) United States
(12) Patent Application Publication
(10) Pub. No.: US 2023/0010422 A1
(43) Pub. Date: Jan. 12, 2023

(54) LEFT/RIGHT BAND

(71) Applicant: Katie McDonough, Osseo, MN (US)

(72) Inventor: Katie McDonough, Osseo, MN (US)

(21) Appl. No.: 17/858,080

(22) Filed: Jul. 6, 2022

Related U.S. Application Data
(60) Provisional application No. 63/218,739, filed on Jul. 6, 2021.

(51) Int. Cl. G09B 19/00

(52) U.S. Cl. CPC G09B 19/00; A61B 90/92

(57) ABSTRACT

The left/right band of the present invention solves the confusion of knowing what is left and what is right on a person's body and knowing the direction of left and right. The left/right band is elastic and includes two free ends configured to be removably fastened together via a fastening member, such as hook and loop fasteners. An identifier is positioned on a top side of the band, wherein the identifier is associated with the left or right side of the user's body. Designed to help a user avoid left/right confusion, the left/right band of the present invention can help young children and people with disabilities, and other users to comprehend the difference between left and right body parts or direction of left and right especially in strenuous, stressful and time sensitive activities.

### CROSS-REFERENCE TO RELATED APPLICATIONS

The present invention claims priority to U.S. Provisional Application Ser.

No. 63/218,739, filed Jul. 6, 2021 entitled “Left/Right Band” which is incorporated in its entirety at least by reference.

### BACKGROUND OF THE INVENTION

### 1. Field of the Invention

The present invention relates to an instructional tool and identifiers and more particularly a left/right band.

### 2. Description of Related Art

In the art of identifiers, there is a captain's band worn on an arm of a user to identify that the user is the captain on the soccer/football team. The band normally has the letter “C” printed on it. This product is used to only identify the team captain and does this with the band with the letter “C” being worn/wrapped around the player's arm. Captain's bands are offered in many color options and all have the letter “C” printed on them. However, a band for identifying directions is needed.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

It is an object of the present invention to help the person wearing the left/right band to understand which side of the body or direction the instructor or other designated person is referencing. It is another object of the prevent invention to be used in surgical settings to help decipher the side of body or body part needing surgery.

In order to do so, in one aspect of the invention, a left/right band is provided, comprising a band comprised of elastic material, the band having a top side, a bottom side, a first free end, and a second free end; a fastening member configured to join the first free end and the second free end; an identifier on the top side or the bottom side associated with a user's left side of the body or right side of the body; and, wherein the left/right band is configured to aid the user in understanding which side of the body an authority user is referencing.

In one embodiment, the fastening member is hook and loop fasteners. In another embodiment, the identifier is text comprising of “L” or “R.” In yet another embodiment, the identifier is a color. In another embodiment, the user's left side of the body or right side of the body is the user's arm or leg. In another embodiment, the user's left side of the body or right side of the body is the user's hand or foot. In one embodiment, the authority user is a coach or teacher.

In another aspect of the invention, a left/right band consists of: an elastic band having a top side, a bottom side, a first free end, and a second free end; hook and loop fasteners configured to join the first free end and the second free end; and, an identifier on the top side associated with a user's left side of the body or right side of the body.

In yet another aspect of the invention, a method is provided, the method for preventing left/right confusion for a user, the method comprising: (a) providing an elastic band having an identifier, the elastic band having a first end and a second end, wherein the first end is configured to be removably attached to the second via a fastening member; (b) positioning the elastic band around a boot of an ice skate worn by a left foot or right foot of a user, such that the band encircles the boot in a center portion of the boot and the first end is attached to the second end via the fastening member encircling a top side and a bottom side of the boot above a blade of the ice skate; and, wherein the identifier is configured to identify either the left foot or the right foot the elastic band is positioned on.

In one embodiment, the identifier is text comprising of “L” or “R.” In one embodiment, the text identifier is positioned on the top side of the boot. In another embodiment, the identifier is a color. In one embodiment, the fastening member is hook and loop fasteners. In one embodiment, the elastic band is comprised of nylon.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a perspective view of the left/right band positioned on a wrist of a user according to an embodiment of the present invention;

FIG. 2 is a top view of the left/right band positioned on an ice skating boot worn by a user according to an embodiment of the present invention;

FIG. 3 is a side perspective view of the left/right band positioned on an ice skating boot worn by a user according to an embodiment of the present invention;

FIG. 4 is a side perspective view of the left/right band positioned on an alternative ice skating boot worn by a user according to an embodiment of the present invention;

FIG. 5 shows top and bottom sides of the left/right band according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide a left/right band.

It is to be understood that the terminology used herein is for the purpose of describing particular embodiments only and is not intended to be limiting. The terms “a” or “an,” as used herein, are defined as to mean “at least one.” The term “plurality,” as used herein, is defined as two or more. The term “another,” as used herein, is defined as at least a second or more. The terms “including” and/or “having,” as used herein, are defined as comprising (i.e., open language). The term “coupled,” as used herein, is defined as connected, although not necessarily directly, not necessarily mechanically, and not permanent. The term “providing” is defined herein in its broadest sense, e.g., bringing/coming into physical existence, making available, and/or supplying to someone or something, in whole or in multiple parts at once or over a period of time. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

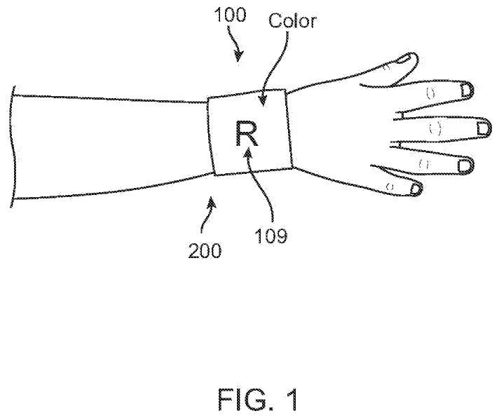

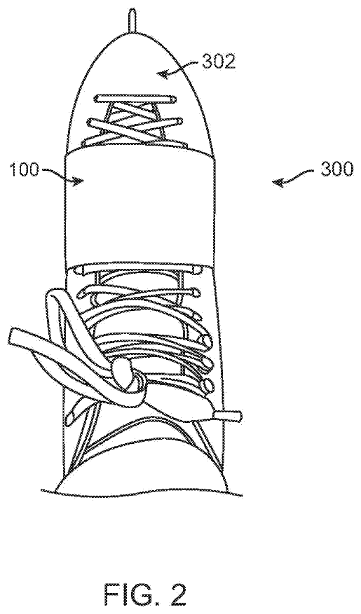

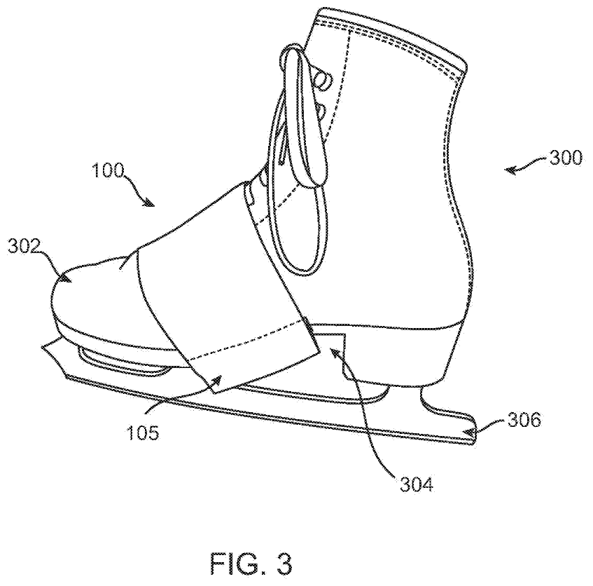

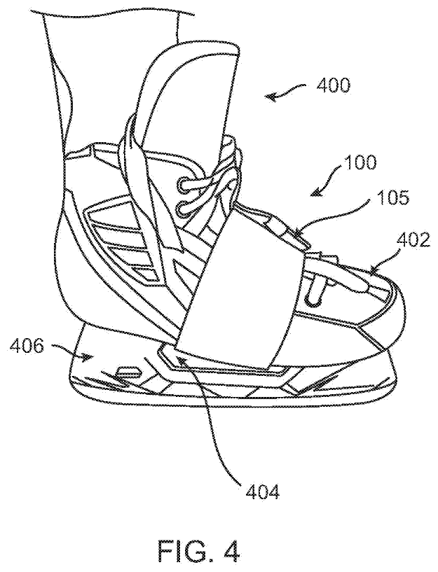

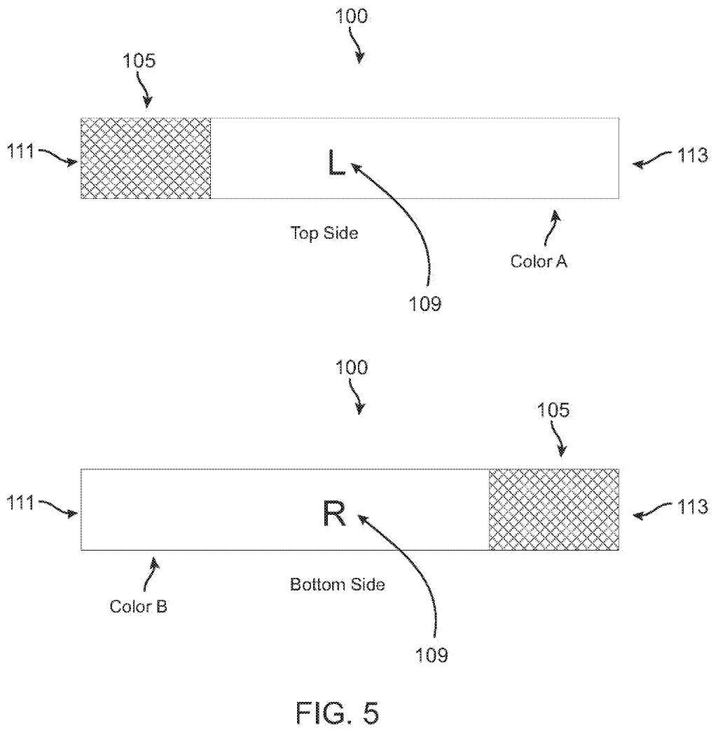

Referring now to any of the accompanying FIGS. 1-5, in some embodiments, the left/right band 100 comprises a strip of elastic material with a fastening member 105. In one embodiment, the fastening member is hook and loop fasteners (such as Velcro®) on each end of the band enabling a user to attach/wrap the band on a mounting location. Hook and loop fasteners are preferred as they offer ease of installation and removal from the mounting location. Other fastening members may be used including but not limited to buttons, zippers, magnets, or similar. It should be understood that a fastening member is required, as the installation of some mounting locations requires the strip of elastic material to have two free ends (111 and 113; FIG. 5). This will be discussed in further details below. Several mounting locations are provided, including but not limited to an arm, hand, wrist, leg, foot, shoe, skate, boot, limb, or object. For example, in FIG. 1 the left/right band 100 of the present invention is installed on a wrist 200 of a user. In FIGS. 2-3, the left/right band 100 of the present invention is installed on an ice skate 300. In FIG. 4, the left/right band 100 of the present invention is installed on a hockey boot 400.

The left/right band of the present invention is provided in a color, which may be any color. The width and length of the band should vary depending on the application, e.g. the band for the skate boot needs to be longer than the band for the wrist or arm. A longer band may be used and the extra portion may be secured to itself via its fastening member. In one embodiment, the left/right band has a width of approximately 3-5 inches, however the width may vary if necessary. During use, a color is used to distinguish between the left and right side of the user's body. In one embodiment, the left/right band comprises the letter “L” and the letter “R” printed on one or each side of the band to give the option of showcasing the letters or just using colors to help decipher left or right. In some embodiments, two different color bands may be used to distinguish what is left and what is right.

For example, a user can have two bands of two different colors, such as one red and one blue. The red color band is placed on the left wrist of the user and blue color band is placed on the right wrist of the user. An authority user, such as parents, teachers, coaches, instructors, baby-sitters, daycare center workers, etc. can call out the colors to help communicate what is the left wrist and what is the right wrist rather than using “left” and “right” which helps combat left-right confusion issues common under stress or time sensitive situations. It is also helpful for young children still learning and associating what is their left side or right side of their body.

Physicians, therapists, aids and other medical care workers can use the bands to help rehab patients and/or to help distinguish what side of body and possibly body part will be worked on during surgery. It is estimated that there are up to 40 worksite surgery errors a week at hospitals and clinics where surgery is not performed at the correct location. The present invention is configured to alleviate these errors.

As previously mentioned, the left/right band of the present invention solves the confusion of knowing what is left and what is right on a person's body and knowing the direction of left and right. The present invention can be used during and for physical activity, various sports activities, in rehabilitation and physical therapy facilities, dementia facilities, retirement communities, assisted living facilities, medical and surgical facilities. The left/right band of the present invention can also help young children and people with disabilities to comprehend the difference between left and right body parts e.g. left/right shoulder/arm vs arm or leg and/or direction of left and right.

In another instance, a user could use the left/right band for dance on their feet or shoes and on wrists/arms. This could help with establishing what foot is left and right and what arm is left and right and also to help the dancer better understand what direction to turn/spin.

It is a particular object of the present invention to be used in hockey and figure skating. Referring now to FIGS. 2-4, the left/right band of the present invention is shown on a figure skate 300 (FIGS. 2-3) and a hockey boot 400 (FIG. 4). In these embodiments, for instance in FIGS. 2-3, a first end is positioned over a top surface 302 of the figure skate 300, and threaded under the bottom surface 304 above the blade 306 of the figure skate 300 and attached to the second end of the band to form a closed loop as illustrated. Similarly, for the embodiment of FIG. 4, a first end is positioned over a top surface 402 of the hockey boot 400, and threaded under the bottom surface 404 above the blade 406 of the hockey boot 400 and attached to the second end of the band to form a closed loop as illustrated. The band should be positioned such that insignia/text “L” or “R” is positioned on the top surface of the figure skate 300 or hockey boot 400. In the embodiments where only a specific color is used, the positioning is not critical, however, the band should wrap around the skate or boot in a center location with the band over the front portion of the top surface skate or boot. For the purposes of the disclosure and claims, the term “identifier” encompasses either the color and or identifying text “L” or “R.”

In these embodiments, the present invention is configured to be used in hockey and figure skating to help skaters better understand what leg/foot they need to be standing on or skating on. For instance, skating is rarely done with two feet on the ice at one time. It's imperative that skaters at all ages/levels have clear understanding of what foot they're using and what foot they will need to transition to. This helps with ease of movement through skating skills, such as crossovers, one foot glides, two foot glides, mohawks, 3-turns, and transitions. As an example, after installing the invention on a user's skate as previously discussed, during use, an authority user is configured to help the user while learning one foot glides by telling the user to pick up their skate with the band on it. This will allow the user to concentrate on other needs during this skill, such as keeping their hands in front of them to improve their balance.

Advantageously, the present invention may be combined with both arms and legs, i.e. a band or bands on a user's arms and a band or bands on a user's shoes, skates, or feet. Referring to skating, or more particularly hockey, in the embodiment as shown in FIG. 1, the present invention is configured to be used on a user's wrist (or glove) to help them understand what arm or hand to use. The present invention may also be used to establish teams in a practice, wherein the team could be split up and one-half wears a color designating right and the other half wears a color designating left.

Referring now to FIG. 5, a top and bottom side of the left/right band 100 is illustrated. In this exemplary instance, fastening members 105 are placed at opposite ends 111 and 113 of the elastic band such that one end on the bottom side can be attached to the other end on the top side. As previously mentioned, in some embodiments, the fastening members, preferably hook and loop fasteners, are configured to attach to the fabric or material the elastic band is comprised of, such as nylon. In these embodiments, the hook and loop fastening member 105 only needs to be placed on one side of the elastic band. Advantageously, in some embodiments the left/right band is reversible, wherein the top side has an identifier 109, such as “L,” with a color (color A), and the bottom side has a different identifier 109, such as “R,” with a different color (color B). In these embodiments, the fastening members 105 may be positioned on both free ends of the left/right band on each side.

In other embodiments, the left/right band may be used in preschool and kindergarten settings to help children learn their left and right hands and feet. The present invention may be used to determine teams for preschool/kindergarten settings by wearing specific colors as previously mentioned.

Multiple sports and forms of exercise use many different muscle groups and require simultaneous concentration. The present invention will help the user (athlete) establish muscle memory at a quicker pace. In baseball, football, track, lacrosse, or other sports and activities, a user could use the present invention to establish what foot the users (players) will lead with for running during exercises and drills. Further, in similar sports, the left/right band of the present invention can be used to establish a dominant arm for kids.

As previously mentioned, in medical surgical settings the present invention is configured to be used and placed on the side of body that is getting surgery and even the particular limb or body part that will be getting worked on in surgery. Also, the present invention could be used in rehabilitation facilities to help patients with dementia, head trauma, or brain surgery to help determine their left and right limbs and directions. Likewise, the present invention could be used in assisted living facilities to help residents with their left/right directions and limbs, while also being placed on objects throughout the facility to help residents better understand directions of different rooms or locations in the facility.

During use, following method may be followed by a user, wherein the method comprises steps (a) providing an elastic band having an identifier, the elastic band having a first end and a second end, wherein the first end is configured to be removably attached to the second end via a fastening member, such as hook and loop fasteners; (b) positioning the band around a boot of an ice skate worn on a foot of the user, such that the band encircles the boot and in the center portion (as shown in FIGS. 2-4) of the boot and the first end is attached to the second end via the fastening member, wherein the identifier is configured to identify which foot (left or right) the elastic band is positioned. In one embodiment, the identifier is a color. In another embodiment, the identifier is text, such an “L” or “R.” In yet other embodiments, the identifier may be a symbol, shape, or design.

It should be understood that the material of the band or strip of elastic material may vary. In some embodiments, the material is nylon which offers natural connection to the hook and loop fasteners. In alternative embodiments, separate loop material is provided to connect with the hooks of the hook and loop fasteners.

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention. It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A left/right band comprising:
a band comprised of elastic material, the band having a top side, a bottom side, a first free end, and a second free end;
a fastening member configured to join the first free end and the second free end;
an identifier on the top side or the bottom side associated with a user's left side of the body or right side of the body; and,
wherein the left/right band is configured to aid the user in understanding which side of the body an authority user is referencing.

2. The left/right band of claim 1, wherein the fastening member is hook and loop fasteners.

3. The left/right band of claim 1, wherein the identifier is text comprising of “L” or “R.”

4. The left/right band of claim 1, wherein the identifier is a color.

5. The left/right band of claim 1, wherein the user's left side of the body or right side of the body is the user's arm or leg.

6. The left/right band of claim 1, wherein the user's left side of the body or right side of the body is the user's hand or foot.

7. The left/right band of claim 1, wherein the authority user is a coach or teacher.

8. A left/right band consisting of:
an elastic band having a top side, a bottom side, a first free end, and a second free end;
hook and loop fasteners configured to join the first free end and the second free end; and,
an identifier on the top side associated with a user's left side of the body or right side of the body.

9. A method for preventing left/right confusion for a user, the method comprising:
(a) providing an elastic band having an identifier, the elastic band having a first end and a second end, wherein the first end is configured to be removably attached to the second via a fastening member;
(b) positioning the elastic band around a boot of an ice skate worn by a left foot or right foot of a user, such that the band encircles the boot in a center portion of the boot and the first end is attached to the second end via the fastening member encircling a top side and a bottom side of the boot above a blade of the ice skate; and,
wherein the identifier is configured to identify either the left foot or the right foot the elastic band is positioned on.

10. The method of claim 9, wherein the identifier is text comprised of “L” or “R.”

11. The method of claim 10, wherein the text identifier is positioned on the top side of the boot.

12. The method of claim 9, wherein the identifier is a color.

13. The method of claim 9, wherein the fastening member is hook and loop fasteners.

14. The method of claim 9, wherein the elastic band is comprised of nylon.
